(19) United States
(12) Patent Application Publication
(10) Pub. No.: US 2022/0313943 A1
(43) Pub. Date: Oct. 6, 2022

(54) VIRTUAL REALITY METHODS AND APPARATUS WHICH USE OR INCLUDE BRAIN ACTIVITY SENSORS

(71) Applicant: Nevermind Capital LLC, Wilmington, DE (US)

(72) Inventors: David Cole, Laguna Beach, CA (US); Hector M. Medina, Laguna Beach, CA (US)

(21) Appl. No.: 17/808,760

(22) Filed: Jun. 24, 2022

Related U.S. Application Data
(63) Continuation of application No. 15/882,997, filed on Jan. 29, 2018, now abandoned.
(60) Provisional application No. 62/478,031, filed on Mar. 28, 2017.

(51) Int. Cl. A61M 21/02; G09B 19/00; G09B 5/04; G09B 5/02; A61B 5/00; A61M 21/00

(52) U.S. Cl. CPC A61M 21/02; G09B 19/00; G09B 5/04; G09B 5/02; A61B 5/486; A61B 5/6803; A61M 21/00; A61M 2021/0027; A61M 2209/088; A61M 2230/10; A61M 2021/0044; A61M 2205/507; A61M 2205/581; A61M 2210/06; A61B 5/291

(57) ABSTRACT

Integrated systems for providing audio, video, light stimulus and/or measuring brain activity through the use of one or more electrodes, e.g., EEG dermatrodes, are described. A system, e.g., a head mounted VR device, for providing stimulation to a user and detecting user responses to said stimulation includes a head mount, a plurality of light emitting elements, e.g., sets of LEDs, a housing secured to said head mount, and at least one or more electrodes for making contact with skin of a user. In various embodiments, the system further determines a user's mental state and provides audio and/or visual feedback to the user.

### RELATED APPLICATIONS

The present application is a continuation of U.S. patent application Ser. No. 15/882,997 filed Jan. 29, 2018, which claims the benefit of U.S. Provisional Patent Application Ser. No. 62/478,031 filed Mar. 28, 2017, both of which are hereby expressly incorporated by reference in their entireties.

### FIELD

The present invention relates to virtual reality methods and apparatus, and more particularly virtual reality methods and apparatus using or including brain activity sensors.

### BACKGROUND

Various self improvement techniques aim to train an individual to be able to control his or her mental state, e.g., to maintain a level of calm even under what some might consider stressful conditions. Brain activity involves electrical activity. The electrical brain activity can be detected using electrodes that are placed in contact with a person's scalp. The detected electrical activity can be processed and interpreted to determine through measurable indicia, e.g., detected electrical brain activity, which portions of a person's brain is active and from this the user's mental state.

Sounds and/or images can be used in an attempt to induce or cause a desired mental state in a user. In some cases a sequence of flashing or pulsed lights, e.g., of one or more different colors, which are flashed on a person's closed eyelids are used to help induce a desired mental state. Such flashing lights can be combined with sounds which may be played to a user while the flashing lights are presented to a user's closed eyelids. The light passing through the closed eyelids can help induce a desired mental state in at least some individuals.

While the use of images, sounds and/or lights to help induce a mental state exist, devices for generating audio and visual stimulus tend to be separate standalone devices from devices, e.g., EEG devices, which are used to measure an individual's mental state. As a result, it is often difficult for an individual to easily control various stimuli while at the same time measuring his/her mental state. For example, while a user may watch a TV or other device to obtain visual input, it may be difficult for a user to also keep attached to his/her scalp one or more electrodes, particularly if the visual input and/or mental measurements are to occur over an extended time period. Also, it is often difficult to quickly attach and remove electrodes, making the process of obtaining the desired stimulus and measurement of brain activity difficult to obtain.

In view of the above it should be appreciated that there is a need for improved methods and apparatus which can be used to apply visual, audio and/or other stimulus to an individual while allowing the user's brain state to be measured. While not necessary for all embodiments it would be desirable if in at least some embodiments the system or device for providing stimulus and/or measuring brain activity could be easily used by an individual without the need for help from others with regard to attaching electrodes and/or measuring brain activity. From a portability and use perspective, it would be desirable if at least in some embodiments a system or device could be implemented in a relatively small and portable form allowing an individual to take the system or device with him or her to a variety of locations and easily use the device or system.

### BRIEF DESCRIPTION OF THE FIGURES

FIG. 1 illustrates an exemplary head mountable system implemented in accordance with one embodiment of the invention.

FIG. 2 illustrates a portion of the system shown in FIG. 1 as viewed from the side which would be seen by a user's eyes when worn by a user.

FIG. 3 shows a lens assembly surrounded by light emitting diodes (LEDs) which can be used as the right eye lens assembly of the system shown in FIGS. 1 and 2.

FIG. 4 illustrates the portion of the system shown in FIG. 2 as viewed from the side which would be seen by a user's eyes when worn by a user but with various internal elements not visible from the outside shown so that the reader can appreciate some of the components included in the exemplary system.

FIG. 5 shows a lens assembly surrounded by LEDs which can be used as the left eye lens assembly of the system shown in FIGS. 1 and 2.

FIG. 6 is a drawing of a flowchart of an exemplary method of controlling a system for providing stimulation, e.g., visual stimulation, to a user, in accordance with an exemplary embodiment.

FIG. 7 is a drawing of a flowchart of an exemplary method of providing stimulation, e.g., visual stimulation to a user, and detecting user responses to said stimulation in accordance with an exemplary embodiment.

FIG. 8 is a drawing of an exemplary assembly of components which may be included in an exemplary system for providing stimulation, e.g., visual stimulation, to a user and/or detecting user responses to said stimulation, in accordance with an exemplary embodiment.

FIG. 9 is a drawing of an exemplary memory which may be included in the head mounted system of FIG. 1, which is shown in FIG. 4, in accordance with an exemplary embodiment.

FIG. 10 is a drawing of an exemplary cell phone, e.g., a smart phone, which may be inserted into a head mount in some embodiments, and its display may be utilized as a display for the head mountable system in accordance with some embodiments.

### SUMMARY

A system for providing audio, video, light stimulus and/or measuring brain activity through the use of one or more electrodes, e.g., EEG dermatrodes, are described. The dermatrodes, in some but not all embodiments, are carbon rubber electrodes which are well suited for making contact with a user's skin layer and sensing electrical activity associated with the brain. A conductive gel or gel layer is used in some embodiments to enhance conductivity and skin contact.

The system can be implemented as a stand alone device, e.g., a head mounted device including one or more processors, memory, display, speakers, LED lights and one or more electrodes. In other embodiments the processor and/or other components are separate from but coupled to, either via wires or wirelessly, the head mounted display device and/or the head-mounted electrodes. In some embodiments the electrodes are secured to the headmount via foam. In at least some such embodiments when placed on a user's head a band or strap supplies tension causing the foam to be compressed and causing the electrodes to make skin contact with the wearer of the head mount to which the various components are mounted or otherwise secured.

Whether implemented as a stand alone device or as a device coupled to a separate processing system, e.g., using a wired or wireless interface, the system is relatively easy to use as compared to known systems where EEG functionality is completely separate from the display system and not integrated on the same mount used to support the display device.

As part of the process of monitoring the user's mental state, the electrodes are used to detect electrical activity in the user's brain. The electrodes are implemented in some embodiments as small, flat or flexible plates made of conductive material, e.g., metal. The electrodes are placed in contact with the user's scalp. As the user's brain cells communicate via electrical impulses, the electrical impulses are detected by the electrodes and reported back to a processor for recording and analysis. Since a user's brain is active all the time, even when the user is asleep the electrical activity at different locations can be detected by different electrodes and used to analyze the user's mental state or plot a chart of detected electrical signals which are often displayed as wavy lines on an EEG recording.

Audio feedback can be, and sometimes is, provided to a user as an indication of the detected mental state. The audio feedback supplied to the user via the speaker or speakers helps the user to understand his or her mental state as interpreted by the processor without the need for the user to open his or her eyes.

A user can put the head mounted display device with electrodes on his or her head where it is secured via a strap, band or other mounting device referred to as a head mount. Each of electrodes contact the user's skin, e.g., forehead, and each electrode senses electrical activity indicative of brain activity corresponding to the area where the electrode makes contact with a user's skin.

The system of the present invention is well suited for use as a cognitive feedback system. In various embodiments the system measures an individual's mental state and provides feedback to the user so that the user can attempt to alter his/her mental state and achieve a desired state, e.g., a calm mental state.

In at least some embodiments the system is for providing stimulation to a user and detecting user responses to said stimulation. The detected response is analyzed and used in some embodiments to provide cognitive feedback in the form of a sound indicative of the user's detected mental state.

The system is easy to use. Furthermore it can be implemented as a compact standalone device making it far easier to use than a system which involves or requires the use of large bulky separate EEG systems in addition to other devices to provide audio and/or visual stimulation.

An exemplary system for providing stimulation to a user and detecting user responses to said stimulation, in accordance with some embodiments, comprises: a head mount; a plurality of light emitting elements; a housing secured to said head mount; and at least one or more electrodes for making contact with skin of a user.

An exemplary method, in accordance with some embodiments, comprises: using a head mounted display to display image content to a user; prompting the user to close the user's eyes; providing visual stimulation by controlling LED light in a preprogrammed sequence while the user's eyes are closed; measuring the user's response to the provided stimulus by detecting electrical signals using electrodes, and providing at least one of audio feedback or visual feedback to the user, said feedback indicative of a mental state determined based on the detected electrical signals.

It should be appreciated that different embodiments can include various combinations of features and that not all features discussed in the summary need be included in all embodiments.

Numerous additional features and embodiments are discussed in the detailed description which follows.

### DETAILED DESCRIPTION

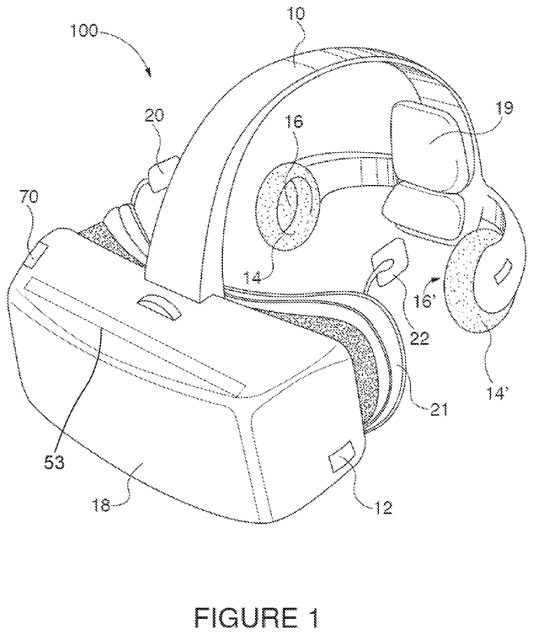

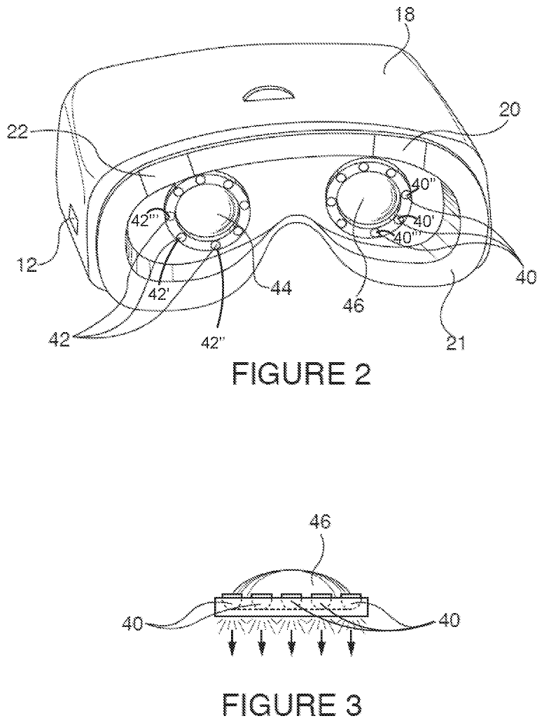

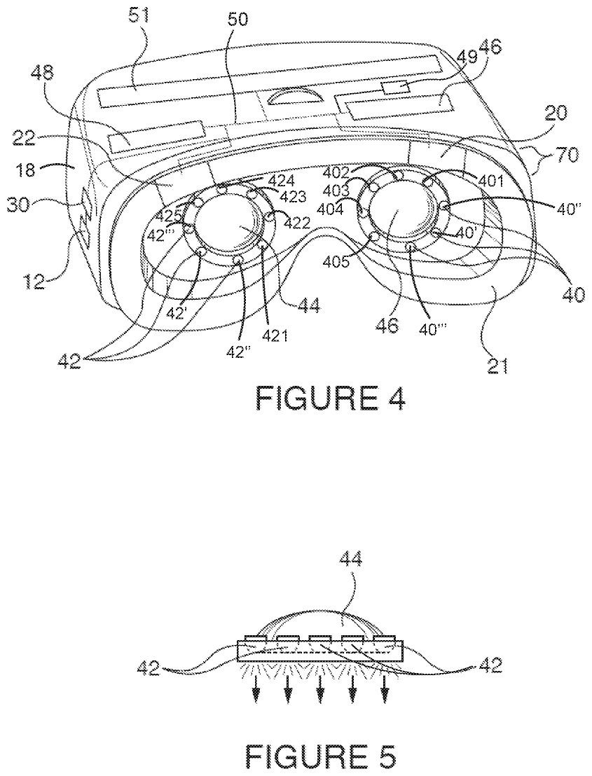

FIG. 1 illustrates an exemplary head mountable system 100 implemented in accordance with one embodiment of the invention. The system 100 includes a head mount 10 which can include a strap or band such as the one shown at the top of the head mount along with a housing 18 in which various elements such as a display 51 are secured along with left eye optics 44 and right eye optics 46 and corresponding light emitting elements (42, 40), respectively. The various elements are secured to the housing 18, e.g., by plastic pieces, clips, screws and/or adhesive which in turn is secured to the head mount 10 using one or more plastic pieces, clips, screws and/or adhesive. The headmount 10 and housing 18 can be integrated, e.g., as a single molded device in some embodiments or with the headmount 10 being made of a different material and being secured to the housing 18 via an adhesive, a clip, snap, screws or some other mechanism. The display device 51, eye optics (44, 46) and light emitting elements ((42′, 42″, . . . , 42′″), (40′, 40″, . . . , 40′″)) are not visible in FIG. 1 but are shown in FIG. 4. An external wired interface 12, such as a USB interface, is included in some embodiments as well as a user input device 70 which, in some embodiments, is a touch pad. The system 100 includes a right electrode 20 and a left electrode 22. While the electrodes (20, 22) are shown in FIG. 1 as extending out from the eye pad 21 used to shield the user's eyes from external light, in some embodiments the electrodes (20, 22) are secured to the foam eye pad 21 as shown in FIG. 2. In some embodiments, system 100 includes both electrodes which are secured to the foam eye pad 21 and electrodes which extend out, e.g. from the eye pad 21. Thus the system 100 can include fixed and/or movable electrodes depending on the embodiment. While two electrodes (right electrode 20, left electrode 22) are shown in FIG. 1, additional electrodes may be used depending on the embodiment with some embodiments using 2, 4, 6, or more electrodes with each electrode corresponding to a different portion of a user's brain. The system shown in FIG. 1 includes a display device 51 which is not visible from the view in FIG. 1 but which can be seen in FIG. 4. The system 100 also includes right speaker 16, left speaker 16′ and ear pads 14, 14′. Head pad or cushion 19 provides padding for the rear of the head mount 10 but can be, and sometimes is, used to secure an additional electrode.

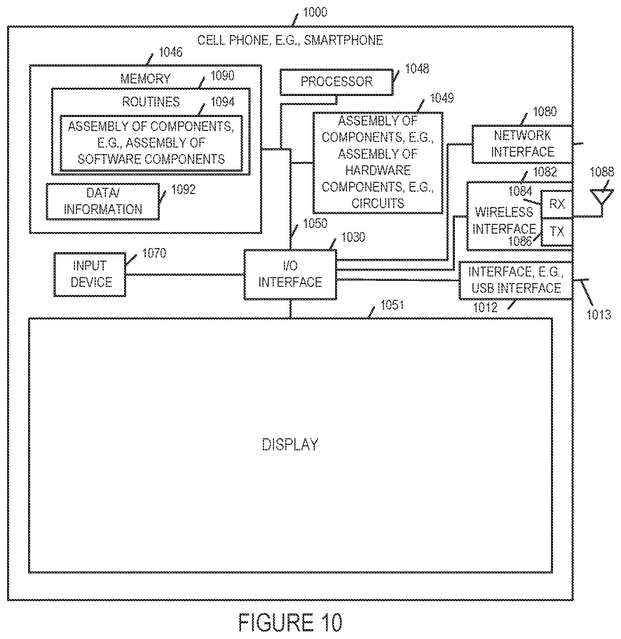

In some embodiments of system 100, the head mount 10 includes a slot 53 in the housing 18 into which a cell phone, e.g., cell phone 1000 of FIG. 10, can be inserted and secured, and the display 1051 of the cell phone 1000, e.g., a smartphone, is used as display 51.

FIG. 2 illustrates a portion of the system shown in FIG. 1 as viewed from the side which would be seen by a user's eyes when worn by a user. In the FIG. 2 illustration right eye lens 46 and left eye lens 44 can be seen surrounded by LEDs ((40′, 40″, . . . , 40′″), (42′, 42″, . . . , 42′″)), respectively, represented as small solid circles. The LEDs ((40′, 40″, . . . , 40′″), (42′, 42″, . . . , 42′″)) may be, and sometimes do, form a sequence of Red, Green and Blue LEDs with each dot representing one LED. Multiple LEDs of each color are included around each lens in some embodiments. For example, in one embodiment, the set 40 of LEDs includes 8 LEDs including, e.g., 3 red LEDs, 3 green LEDs and 2 Blue LEDs, and the set 42 of LEDs includes 8 LEDs including, e.g., 3 red LEDs, 3 green LEDs and 2 blue LEDs. In another embodiment, a set 40 of LEDs includes 9 LEDs including 3 red LEDs, 3 green LEDs and 3 Blue LEDs, and a set 42 of LEDs includes 9 LEDs including 3 red LEDs, 3 green LEDs and 3 Blue LEDs. In some embodiments, LEDs in a set of LEDs, e.g., corresponding to a lens, alternate between the various colors being used in the set as traversing the set in a clockwise or a counter-clockwise direction, e.g., Red, Green, Blue, Red, Green, Blue, Red, Green, Blue. The LEDs (42′, 42″, . . . , 42″′) for the left eye and the LEDs right eye (40′, 40″, . . . , 40′″) can be, and sometimes are, controlled separately. In some embodiments, each individual LED in a set of LEDs, e.g., a right eye LED set or a left eye LED set, can be, and sometimes is, individually controllable. In various embodiments, LED control includes output intensity level control and control of the time intervals at which an LED is On or Off. In some embodiments, different color LEDs are controlled to be on at different times. In some embodiments, particular selected LEDs are controlled to flash, e.g., at predetermined controlled rates. In some embodiments, particular selected LEDs are controlled to flash, e.g., at a predetermined frequency. In some embodiments, selected LEDs are controlled to be on and off to create a light pattern. IN some embodiments, the created light pattern is a time varying light pattern. In some embodiment, the created light pattern is a rotating light pattern or a moving light pattern.

FIG. 3 shows a lens assembly 46 surrounded by LEDs (40′, 40″, . . . , 40′″), which form a set of lighting elements 40, and the lens assembly 46 and the set of lighting elements 40 can be used as the right eye lens assembly 46 and surrounding set of lighting elements 40 of the system 100 shown in FIGS. 1, 2 and 4.

FIG. 4 illustrates the portion of the system shown in FIG. 2 as viewed from the side which would be seen by a user's eyes when worn by a user but with various internal elements not visible from the outside shown so that the reader can appreciate some of the components included in the exemplary system. In FIG. 4, display 51, processor 48, memory 46, assembly of components 49, e.g., an assembly of hardware components, e.g., circuits, interface 12, I/O interface 30 and user input device 70, e.g., a touchpad, of system 100 are shown. Display 51, processor 48, memory 46, assembly of components 49 and I/O interface 30 are, in some embodiments, included within housing 18. In some embodiments, the display 51, processor 48, memory 46, and assembly of components 49 are coupled together via a bus 50. In some embodiments, the display 51, processor 48 and memory 46 are included as part of an external device, e.g., a smart cell phone, which is inserted into a slot 53 in the housing 18 and secured within the housing 18. In the case of an external processor being supplied with the detected brain electrical signals detected by the electrodes 20, 22, the signals can be supplied via interface 12, e.g., a USB interface, which is coupled to the bus 50 via input/out (I/O) interface 30. In FIG. 4, each of the lighting elements in the set of lighting elements 40, which surround right eye lens 46, has been identified with a reference number. Set of lighting elements 40 includes LED 40′, LED 40″, LED 401, LED 402, LED 403, LED 404, LED 405, LED 40′″. In FIG. 4, each of the lighting elements in the set of lighting elements 42, which surround left eye lens 44, has been identified with a reference number. Set of lighting elements 42 includes LED 42, LED 42′, LED 421, LED 422, LED 423, LED 424, LED 425, LED 42″′.

In some embodiments, the set 40 of LEDs includes lighting elements of multiple different colors, said multiple different colors including two or more of red, green or blue. In some embodiments, the set 42 of LEDs includes lighting elements of multiple different colors, said multiple different colors including two or more of red, green or blue. For example, in one embodiment LED 40′, 401, LED 403 and LED 405 are red color LEDs; LED 40″, LED 402, LED 404 and LED 40′″ are green LEDs; LED 42′, LED 421, LED 423 and LED 425 are red color LEDs; and LED 42″, LED 422, LED 424 and LED 42′″ are green LEDs. In another embodiment LED 40′, LED 402 and LED 405 are red color LEDs; LED 40″, LED 403 and LED 40′″ are green LEDs; LED 401 and LED 404 are blue LEDs; LEDs 42′, LED 422 and LED 425 are red color LEDs; LED 42″, 423 and LED 42′″ are green LEDs; and LEDs 421 and LED 424 are blue LEDs;

In some embodiments, lighting element set 40 includes 9 or more LEDs. In some embodiments, lighting element set 42, includes 9 or more LEDs. In some embodiments, the set of lighting elements 40 includes the same number of lighting elements as the number in the set of lighting elements 42. In some embodiments, the LED color pattern in set 40 is the same the LED color pattern of set 42. In some embodiments, the LED color pattern in set 40 a mirror image of the LED color pattern of set 42.

FIG. 5 shows a lens assembly 44 surrounded by LEDs (42′, 42″, . . . , 42′″), which form a set of lighting elements 42, which can be used as the left eye lens assembly 44 and surrounding set of lighting elements 42 of the system shown 100 in FIGS. 1, 2 and 4.

In some embodiments, an exemplary system integrates EEG electrodes, e.g., dermatrodes, e.g., 2-channel EEG dermatrodes, with corresponding amplifiers and LEDs, e.g., 2 sets of high-intensity red, green and blue (RGB) LEDs, into an all-in-one headset, e.g., an all-in-one virtual reality (VR) headset. In some embodiments, the exemplary system supports frequency-follow-response brainwave entrainment, EEG monitoring/Biofeedback and Virtual Reality into a single device. In various embodiments, the exemplary system can, and sometimes does, induce brainwave state changes and then provide relevant VR cognitive training programming. In some embodiments, the exemplary system can, and sometimes does, monitor for brainstate and create logical bridges in VR programming. In some embodiments, the exemplary system can, and sometimes does, monitor and reinforce brainstates such as relaxation, focus, or hyper-alertness. In some embodiments, the system can be, and sometimes is, used to de-program anxiety. Various embodiments, of the exemplary system are well suited for patient distraction therapy and VR phobic therapies. In some embodiments, the exemplary system supplements VR visual programming with hypnagogic imagery generated by LEDs.

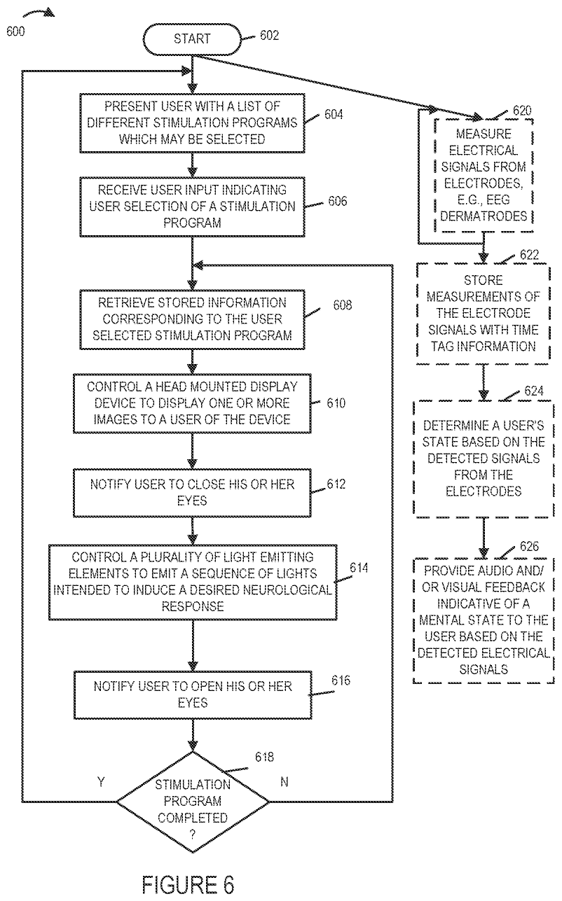

FIG. 6 is a drawing of a flowchart 600 of an exemplary method of controlling a system for providing stimulation, e.g., visual stimulation, to a user, in accordance with an exemplary embodiment. In some embodiments, the system implementing the method of flowchart 600 is a single apparatus, e.g., head mounted VR apparatus 100 of FIG. 1. In some such embodiments, the head mounted VR apparatus 100 includes a processor 48, memory 46, a display 51, and two sets of LEDs, e.g., 2 sets (42, 40) of high intensity RGB LEDs, one set per eye. Operation starts in step 602 in which the system 100 is powered on and initialized and proceeds to step 604. In some embodiments, operation also proceeds from step 602 to step 620.

In step 604 the system presents a user with a list of different stimulation programs which may be selected. For example, processor 48 controls head mounted VR device 100 to present, on display 51, a list of stimulation programs which may be selected by the user wearing head mounted VR device 100. In one embodiment, the list of different stimulation programs includes two or more of: a VR cognitive training program, a relaxation program, a focus program, a hyper-alertness program, an anxiety de-programming program, a distraction therapy program, and a VR phobic therapy program. In one embodiment, the list of different stimulation programs includes three or more of: a VR cognitive training program, a relaxation program, a focus program, a hyper-alertness program, an anxiety de-programming program, a distraction therapy program, and a VR phobic therapy program. In some embodiments, the list of different programs includes at least one customized program customized for the user. In some embodiments, the list of different programs includes multiple customized programs of the same type, e.g. multiple customized relaxation programs, corresponding to different users. Operation proceeds from step 604 to step 606.

In step 606 the head mounted VR system 100 receives user input indicating a user selection of a stimulation program. For example, user input device 70, e.g., a touch pad, receives user input indicating the user selection of one of the plurality of programs presented in step 604, and processor 48 which is coupled to user input device 70 monitors for and detects user input received via user input device 70. Operation proceeds from step 606 to step 608. In step 608 the system retrieves stored information corresponding to the user selected stimulation program. For example, in step 608 processor 48 retrieves from memory 46 a pre-stored set of information corresponding to the selected user stimulation program, said set of information including: i) a set of images corresponding to the left eye, ii) an a set of images corresponding to the right eye, iii) control information for implementing a first LED light sequence to stimulate the left eye; and iv) and control information for implementing a second LED light sequence to stimulate the right eye. Operation proceeds from step 608 to step 610.

In step 610 a head mounted display device is controlled to display one or more images to a user of the device. For example, processor 48 controls head mounted VR apparatus 100 to display one or more images on display 51 to a user wearing head mounted VR device 100. In some embodiments, the displayed one or more images are images which were previously stored in memory 46, e.g. one or more images corresponding to a pre-stored programs, and retrieved in step 608 based on the user selection received in step 606. In some embodiments, a right portion of the display is used to display a set of images corresponding to the right eye, and a left part of the display is used to display a set of images corresponding to the left eye. Operation proceeds from step 610 to step 612.

In step 612 the system notifies the user to close his or her eyes. In some embodiments, processor 48 controls display 51 to display a message notifying the user wearing device 100 to close his or her eyes. In some other embodiments, processor 48 controls speakers 16, 16′ to output an audio message to the user to close his or her eyes. Operation proceeds from step 612 to step 614.

In step 614 a plurality light emitting elements are controlled to emit a sequence of lights intended to induce a desired neurological response. For example, processor 48 controls left eye LEDs 42 and/or right eye LEDs 44 of head mounted VR apparatus 100 to emit one or more sequences of light while the user is wearing head mounted VR device 100. In some embodiments, information used to generate the one or more sequences of light was previously stored in memory 46, e.g. a particular sequence of light for each eye corresponding to a particular pre-stored program, and retrieved in step 608 based on the received user input of step 606. Operation proceeds from step 614 to step 616.

In step 616 the system notifies the user to open his or her eyes. In some embodiments, processor 48 controls speakers 16, 16′ to output an audio message to the user to open his or her eyes. Operation proceeds from step 616 to step 618.

In step 618 the system determines if the user selected stimulation program is complete. If the determination of step 618 is that the stimulation program is not complete, then operation proceeds from step 618 to step 608, in which additional stored information corresponding to the stimulation program is retrieved, e.g., for a subsequent segment of the user selected stimulation program which is a multi-segment stimulation program. If the determination of step 618 is that the stimulation program is complete, then operation proceeds from step 618 to step 604, in which the system presents the user with a list of different stimulation programs which may be selected. In some embodiments, the list presented to the user in step 604 during different iterations of step 604 may be, and sometimes is different. In some embodiments, the presented list is based on a determined current user's state. In some such embodiments, the determined current user's state is based on detected electrical electrode, e.g. EEG dermatrode, signal responses to visual stimulation, e.g., visual stimulation of steps 610 and/or steps 614.

In some embodiments, steps 612 and 616 are omitted. In some such embodiments, steps 610 and 614 are at least partially overlapping. In some embodiments, the intensity output of the LEDs are controlled to be at lower maximum level when it is expected that the user with have his or her eyes open and are controlled to be at higher maximum level when it is expected that the user with have his or her eyes closed. In some embodiments, the controlled maximum LED intensity output levels are custom levels corresponding to a particular user.

Returning to step 620, in step 620 electrical signals from electrodes, e.g., from a plurality of EEG dermatrodes, are measured. For example electrical signals from electrodes 20, 22 in VR apparatus 100 are measured. Step 620 is performed on an ongoing basis, e.g., repetitively, including time intervals in which the user is being subjected to visual stimulation. Operation proceeds from step 620 to step 622. In step 622 the measurements of the detected electrode signals are stored along with time tag information. The recorded time tag information allows the detected brain activity to be correlated to the stimulation, e.g., visual stimulation to which the user is being subjected as part of the selected stimulation program. Operation proceeds from step 622 to step 624. In step 624 the user's state, e.g., user's current state, is determined based on the detected signals from the electrodes and the visual stimulation to which the user is subjected. Operation proceeds from step 624 to step 626. In step 624 audio and/or visual feedback indicative of a mental state of the user is provided to the user based on the detected electrical signals. In some embodiments, the processor sends a message of the determined mental state to a display and displays the message to the user. In some embodiments the processor generates and sends an audio indication or audio message to the user indicative of mental station which is output via speakers 16, 16′. In various embodiments, one or more determinations of step 624 are used as input to decide if the stimulation program is completed in step 618 and/or to determine a list to be presented in a particular iteration step 604. In some embodiments, a particular LED light pattern is used to indicate determined mental state. In various embodiments, steps 622, 624 and 626 are performed multiple times. For example, based on a collected set of electrode measurements, e.g., over a predetermined time interval, a user mental state determination is made, and then audio and/or visual feedback indicative of the user's mental state is provided to the user.

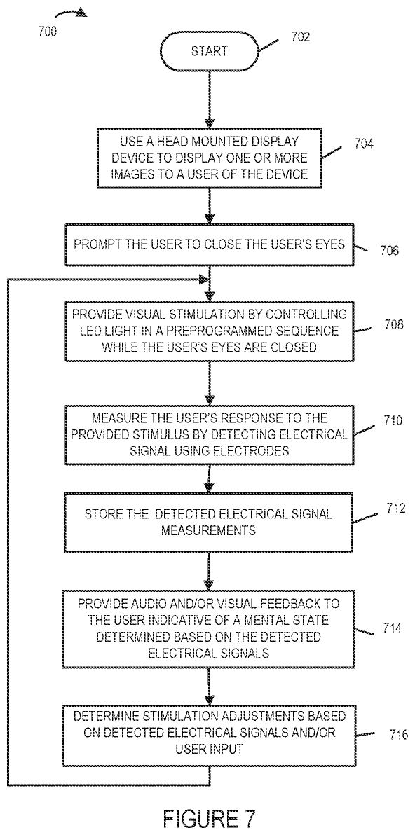

FIG. 7 is a drawing of a flowchart 700 of an exemplary method of providing stimulation, e.g., visual stimulation to a user, and detecting user responses to said stimulation in accordance with an exemplary embodiment. The method of flowchart 700 is, e.g., implemented by head mounted VR device 100 of FIG. 1. Operation starts in step 702 in which a system, a head mounted VR apparatus such as head mounted VR device 100 of FIG. 1, is powered on and initialized. Operation proceeds from step 702 to step 704.

In step 704 a head mounted display device, e.g., headmounted VR device 100, is used to display, e.g., on display 51, one or more images to a user of the device. Operation proceeds from step 704 to step 706 in which a user is prompted to close the user's eyes. In one embodiment, the prompt may be via a message generated by and sent from processor 48 to display 51. In another embodiment, the prompt may be via an audio message generated by and sent from processor 48 to speakers 16, 16′, which output the audio message. Operation proceeds from step 706 to step 708.

In step 708, visual stimulation is provided by controlling LED light in a preprogrammed sequence while the user's eyes are closed. For example, processor 48 controls LEDs, e.g., high intensity RGB LEDs, to be on/off, in a preprogrammed sequence while the user's eyes are closed. In some embodiments, different color LEDs within a set of LEDs 42, 40, are controlled to be on at different times in the preprogrammed sequence. In some embodiments, the LED output intensity of an LED determined to be on is controlled to be at different levels at different times in the sequence. In some embodiments, during at least a portion of the sequence an LED corresponding to a right eye is controlled to be on at a different time in the sequence than corresponding LED corresponding to the left eye. In some embodiments, LED output intensity levels for each eye are controlled independently, e.g., with custom output right and left eye intensity levels being set for a particular user, e.g. in response to a determined user response sensitivity for each user eye. Operation proceeds from step 708 to step 710.

In step 710 the user's response to the provided stimulus is measured by detecting electrical signal using electrodes, e.g., EEG dermatrodes. In some embodiments, the electrodes are part of a wearable system including a headmounted display. In some embodiments, the electrodes include first and second electrodes which contact different parts of the user's head, e.g., different parts of the user's forehead or right and left temple areas, providing separate electrical channels used to monitor the user's brain activity. For example, electrodes 22, 20 of device 100, which are in contact with the skin of the user, detect electrical signals, e.g., user brainwave signals. In some embodiments, each of the electrodes 22, 20 include an embedded analog amplifier, for amplifying a low level detected brain wave signal to produce an amplified received brainwave signal, and an embedded A/D circuit for converting the amplified received brainwave signal to a digital signal which is input to processor 48. In some embodiments, the amplifiers and A/D convertors are standalone devices included in device 100 between the electrodes 22, 20 and the processor 48. Operation proceeds from step 710 to step 712.

In step 712 the detected electrical signal measurements are stored, e.g., in a record corresponding to the user and further including information indicating the visual stimulation to which the user was subjected and time tag information for correlating the detected responses to the visual stimulation. Operation proceeds from step 712 to step 714.

In step 714 audio and/or visual feedback is provided to the user indicative of a mental state determined based on the detected electrical signals. For example, in step 714 the processor 48 determines a user's state based on the received measurements of step 710, e.g., comparing received measurements to store measurement profile information corresponding to different alternative user mental states to find a best match. Then, processor 48 generates and sends feed back information to the user to be communicated to the user via a visual message via the display 51 or via an audio message or indication, via speakers 16, 16′. In some embodiments, different tones, different tone patterns or different tone spacing is used to indicate different mental states. In some embodiments, providing audio or visual feedback to the user indicative of a mental state determined based on the detected electrical signals includes varying an audio signal output as a function of a first electrical signal detected by an electrode, e.g., said first electrode. In some embodiments, providing audio or visual feedback to the user indicative of a mental state determined based on the detected electrical signals includes varying a visual output as a function of an electrical signal, e.g., a first electrical signal, detected by an electrode, e.g., said first electrode, said visual output being an image or color displayed on said head mounted display or one or more lights mounted in said head mounted display, said one or more lights being separate from said visual display. Operation proceeds from step 714 to step 716.

In step 716 the system determines stimulation adjustments based on the detected electrical signals and/or user input. In some embodiments, in step 716 the system generates a new, e.g., slightly modified, preprogrammed sequence of LED light stimulation. In some embodiments, the adjustments are refinements to a baseline predetermined LED light stimulation sequence, said adjustments being based on the detected measured user responses via electrode measurements, and multiple iterations of adjustment are performed, e.g., in a closed loop control manner. Operation proceeds from step 716 to step 708.

In step 708 the system provides visual stimulation by controlling LED light in accordance with the determined stimulation adjustments of step 716, e.g., in accordance with the new preprogrammed sequence of LED light stimulation.

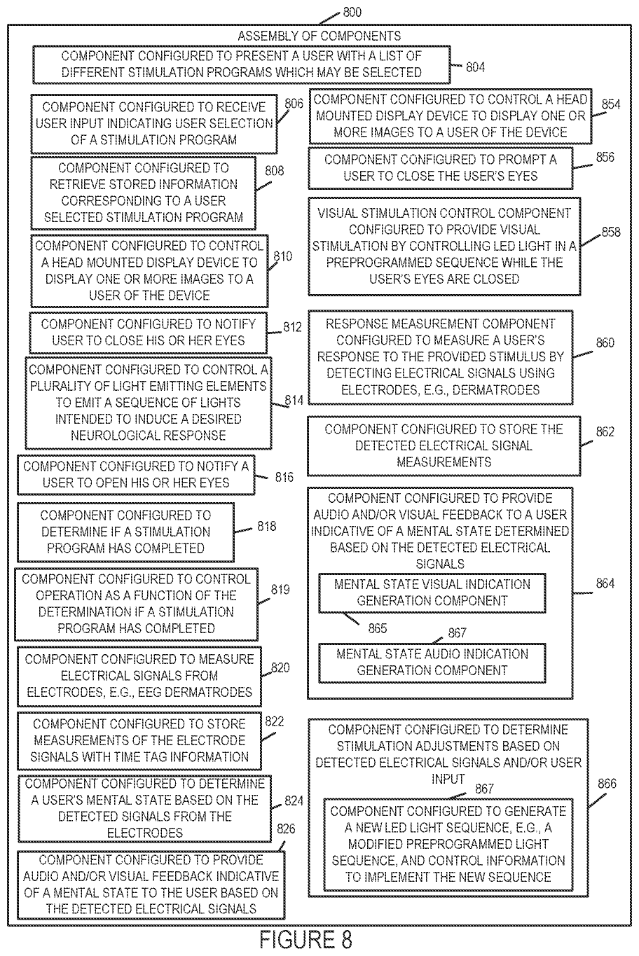

FIG. 8 is a drawing of an assembly of components 800, in accordance with an exemplary embodiment. In some embodiments, assembly of components 800 is included in an exemplary system for providing stimulation, e.g., visual stimulation, to a user and/or detecting user responses to said stimulation, in accordance with an exemplary embodiment, e.g., head mountable system 100, e.g., head mountable device, of FIG. 1.

The components in the assembly of components 800 can be, and in some embodiments are, implemented fully in hardware within the processor 48, e.g., as individual circuits. The components in the assembly of components 800 can, and in some embodiments are, implemented fully in hardware within the assembly of components 49, e.g., as individual circuits corresponding to the different components. In other embodiments some of the components are implemented, e.g., as circuits, within the processor 48 with other components being implemented, e.g., as circuits within assembly of components 49, external to and coupled to the processor 48. As should be appreciated the level of integration of components in the processor 48 and/or with some components being external to the processor 48 may be one of design choice.

Alternatively, rather than being implemented as circuits, all or some of the components 800 may be implemented in software and stored in the memory 46 of the system 100, with the components controlling operation of system 100 to implement the functions corresponding to the components when the components are executed by a processor, e.g., processor 48. In some such embodiments, the assembly of components 800 is included in the memory 46 as assembly of components, e.g., an assembly of software components. In still other embodiments, various components in assembly of components 800 are implemented as a combination of hardware and software, e.g., with another circuit external to the processor providing input to the processor 48 which then under software control operates to perform a portion of a component's function. While shown in the FIG. 4 embodiment as a single processor 48, e.g., computer, it should be appreciated that the processor 48 may be implemented as one or more processors, e.g., computers.

When implemented in software the components include code, which when executed by the processor 48, configure the processor 48 to implement the function corresponding to the component. In embodiments where the assembly of components 800 is stored in the memory 46, the memory 46 is a computer program product comprising a computer readable medium comprising code, e.g., individual code for each component, for causing at least one computer, e.g., processor 48, to implement the functions to which the components correspond. Completely hardware based or completely software based components may be used. However, it should be appreciated that any combination of software and hardware, e.g., circuit implemented components may be used to implement the functions. As should be appreciated, the components illustrated in FIG. 1 control and/or configure the system 100, e.g., a head mountable device or elements therein such as the processor 48, to perform the functions of corresponding steps illustrated in the method of the flowchart of FIG. 6, FIG. 7 and/or described with respect to any of the Figures. Thus the assembly of components 800 includes various components that perform functions of corresponding steps of one or more of FIG. 6 and/or FIG. 7.

FIG. 8 is a drawing of an exemplary assembly of components 800 which may be included in an exemplary system for providing stimulation, e.g., visual stimulation, to a user and/or detecting user responses to said stimulation, in accordance with an exemplary embodiment. Assembly of components 800 includes a component 804 configured to present a user with a list of different stimulation programs which may be selected, a component 806 configured to receive user input indicating user selection of a stimulation program, a component 808 configured to receive stored information corresponding to a user selected stimulation program, a component 810 configured to control a head mounted display device to display one or more images to a user of the device, a component 812 configured to notify a user to close his or her eyes, a component 814 configured to control a plurality of light emitting elements to emit a sequence of lights intended to induce a desired neurological response, a component 816 configured to notify a user to open his or her eyes, a component 818 configured to determine if a stimulation program has completed, and a component 819 configured to control operation as a function of the determination if a stimulation program has completed. Assembly of components 800 further includes a component 820 configured to measure electrical signals from electrodes, e.g., from a plurality of EEG dermatrodes, a component 822 configured to store measurement information of the electrode signals with time tag information, a component 824 configured to determine a user's mental state based on the detected signals from the electrodes, and a component 826 configured to provide audio and/or visual feedback to the user based on the detected signals.

Assembly of components 800 further includes a component 854 configured to control a head mounted display device to display one or more images to a user of the device, a component 856 configured to prompt a user to close the user's eyes, a visual stimulation control component 858 configured to provided visual stimulation by controlling LED light in a preprogrammed sequence while a user's eyes are closed, a response measurement component 860 configured to measure a user's response to provided stimulus by detecting electrical signal using electrodes, e.g., dermatrodes, a component 862 configured to store the detected electrical signal measurements, a component 864 configured to provide audio and/or visual feedback to a user indicative of a mental state determined based on detected electrical signals, and a component 866 configured to determine stimulation adjustments based on detected electrical signals and/or user input. Component 864 includes a mental state visual indication generation component 865 configured to generate a visual indication, e.g., a message, symbol, icon, and/or color indication, to communicate a determined mental state, and a mental state audio indication generation component 867 configured to generate an audio indication, e.g. audio message, tone, tone pattern, tone spacing, or audio output level, to communicate a determined mental state. Component 866 includes a component 867 configured to generate a new LED light sequence, e.g., a modified preprogrammed light sequence, and control information to implement the new LED light sequence.

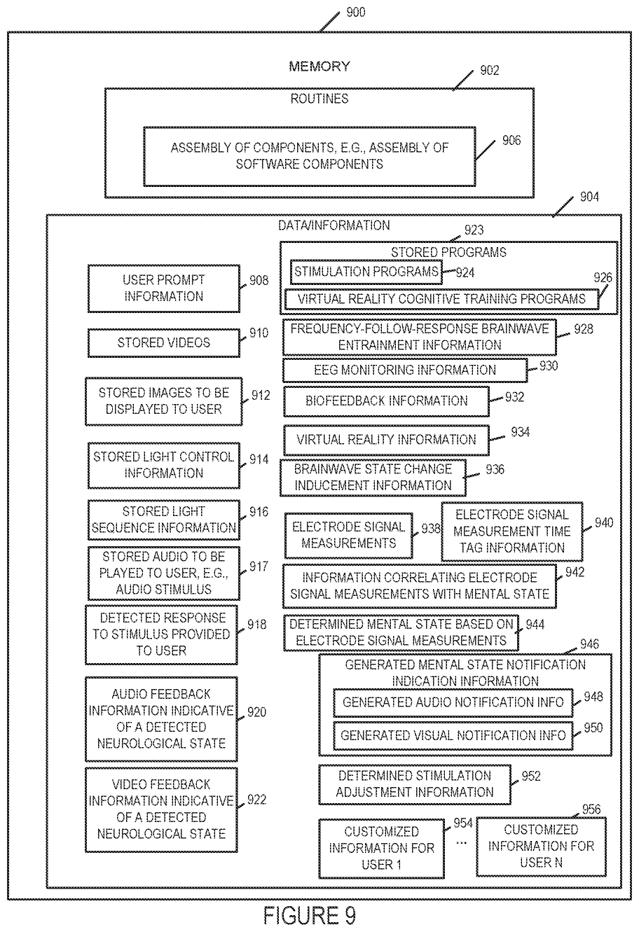

FIG. 9 is a drawing of an exemplary memory 900 in accordance with an exemplary embodiment. Exemplary memory 900 is, e.g., memory 46 of head mounted system 100 of FIG. 1 which is shown in FIG. 4. Memory 900 includes routines 902 and data/information 904. Routines 902 include assembly of components 906, e.g. assembly of software components, e.g., an assembly of software modules.

Data/information 904 includes user prompt information 908, stored videos 910, stored images to be displayed to a user 912, stored light control information 914, stored light sequence information 916, stored audio to be played to a user, e.g., audio stimulus 917, detected response to stimulus, e.g., visual and/or audio stimulus, provided to a user 918, audio feedback information indicative of a detected neurological state 920, video feedback information indicative of a detected neurological state 922. Exemplary user prompts 908 include prompts to select a stored stimulation program and prompts to close ones eyes. Stored videos 910 and images 912, in some embodiments, include different sets of images to be displayed for left and right eyes. Stored videos 910, stored images 912, stored light control information 914, e.g., LED light control information, and stored light sequence information, 916, e.g., LED light control information, is used to generated images and activate LEDs to visually stimulate the user.

Data/information 904 further includes stored programs 923 including stimulation programs 924 and virtual reality cognitive training programs 926, frequency-follow-response brainwave entrainment information 928, EEG monitoring information 930, virtual reality information 934, and brainwave state change inducement information 936. Exemplary stored programs 923 include relaxation monitoring and reinforcement programs, focus monitoring and reinforcement programs, hyper-alertness monitoring and reinforcement programs, anxiety de-programming programs, patient distraction therapy programs, and VR phobic therapy programs.

Data/information 904 further includes electrode signal measurements 938, electrode signal measurement time tag information 940, information correlating electrode signal measurements with mental state 942, determined mental state based on electrode signal measurements 944, generated mental state notification indication information 946, determined simulation adjustment information 952, and customized information for a plurality of users (customized information for user 1 954, . . . , customized information for user N 956). Electrical signals detected by different electrodes, e.g. electrode 20 vs electrode 22, at different locations corresponding to different portions of the brain may have different significance with regard to mental state, and correlation information for processing and/or interpreting the received signals from the different electrodes, e.g., 20 and 22, is includes in information 942.

Generated mental state notification indication information 946 includes generated audio notification information 948 and generated visual notification information 950. In some embodiments, the generated audio notification information 948 is an audio message indicating the determined mental state, which is to be output to the user, e.g., via speakers 16, 16′. In some embodiments, the generated audio notification information 948 is a particular selected tone to be output to the user via speakers 16, 16′, different tones correspond to different determined mental states. In some embodiments, the generated audio notification information 948 is a particular selected pattern of tones to be output to the user via speakers 16, 16′, different patterns of tones correspond to different determined mental states. In some embodiments, the generated audio notification information 948 is a particular selected tone temporal spacing to be output to the user via speakers 16, 16′, different tone temporal spacing corresponding to different determined mental states. In some embodiments, the generated visual notification information 950 is a visual message to the user presented on display 51 indicating a determined mental state. In some embodiments, the generated visual notification information 950 is a particular color displayed on a portion of the display 51, different colors used to indicate different mental states. In some embodiments, the generated visual notification information 950 is a particular controlled LED light output on LED sets (40, 42), e.g., with different colors indicating different mental states, with different flashing rates indicating different mental states, or with different intensities indicating different mental states.

FIG. 10 is a drawing of an exemplary cell phone 1000, e.g., a smartphone, in accordance with an exemplary embodiment. Exemplary cell phone 1000 includes a processor 1048, a memory 1046, an assembly of components 1049, e.g., an assembly of hardware components, e.g., circuits, and an I/O interface 1030 coupled together via a bus 1050 over which the various elements can interchange data and information. Exemplary cell phone 1000 further includes an input device 1070, e.g., a keypad, a display 1051, a network interface 1080, a wireless interface 1082, an interface 1012, e.g., a USB interface, coupled to I/O interface 1030, via which the various devices (1070, 1051, 1080, 1082, and 1012) are coupled to bus 1050. Wireless interface 1082 includes a wireless receiver 1084 and a wireless transmitter 1086. The receiver 1084 and transmitter 1086 are coupled to antenna 1088. Memory 1046 includes routines 1090 and data/information 1092. Routines 1090 includes assembly of components 1094, e.g., an assembly of software components.

In some embodiments, cell phone 1000 is inserted into a slot, e.g., slot 53, into a head mount, e.g., head mount 10 of system 100 of FIG. 1. In some such embodiments, display 1051 of cell phone 100 serves as display 51 in system 100. In some embodiments, the electrodes (20, 22) of system 100 are coupled, e.g., via bus 50, I/O interface 30, interface 12, cable 1013, interface 1012, I/O interface 1030 and bus 1050, to processor 1048 to monitor electrical signals indicative of brain activity and provide the user of system 100 one of audio or visual feedback indicative of a detected neurological state.

In some embodiments, one or more of the components, in assembly of components 800 of FIG. 8 is included in processor 1048. In some embodiments, one or more of the components, in assembly of components 800 of FIG. 8 are included in assembly of components 1049. In some embodiments, one or more of the components, in assembly of components 800 of FIG. 8 are included in assembly of components 1094 in memory 1046.

In some embodiments, one or more of the elements in data/information 904 is included in data/information 1092.

In some embodiments an exemplary system, e.g., system 100, includes a head mount 10; a plurality of light emitting elements 40, 42, e.g., Red, Green and/or Blue (RGB) LEDs surrounding each of left eye lens 44 and right eye lenses 46, at least one or more electrodes 20, 22 secured to said head mount 10 for making contact with skin of a user; an audio output device, e.g., one or more speakers 16, 16′, is secured to the head mount 10; and a first processor 48, said first processor 48 being configured to control the plurality of light emitting elements 40, 42 to display a sequence of lights during a first period of time.

In some embodiments the system 100 further includes a head mounted display device 51. The display device 51 can be integrated into the system or a cell phone, e.g., cell phone 1000, may be inserted into a slot 53 in the head mount 10 which is used to hold the cell phone with the cell phone's display then being used as the display 51. The system 100 also includes the processor 48 and memory 46 which are coupled together by bus 50 as shown in FIG. 4. The processor 48 and memory 46 may be part of a cell phone in embodiments where a cell phone display is used. For example processor 48 may be processor 1048 and memory 46 may be memory 1046 of cell phone 1000. However in other embodiments the processor 48 and memory 46 are part of an integrated head mounted system 100 such as the one shown in FIG. 1. Left electrode 22 and right electrode 20 may be connected to the bus 50 via wires. In some embodiments the electrodes extend out from the system and can be placed on a user's temples after the head mount is placed on the user's head. See, for example the FIG. 1 embodiment. In other embodiments rather than being left loose, the electrodes 20, 22 are mounted to a foam or other soft compressible material which form a seal around the user's eyes as shown in FIG. 4. In such an embodiment when placed on a user's head the electrodes 20, 22 will come into contact with the user's skin and be able to sense electrical signals, e.g., indicative of a user's brain activity. A conductive gel coating may be, and sometime is, coated onto the electrodes 20, 22 before the head mounted device is secured to a user's head to improve electrical sensitivity and contact between the user's skin and the electrodes 20, 22. While two electrodes 22, 20 are shown providing at least left and right channels for sensing brain activity additional electrodes can be included and secured to other locations on a user's head to detect brain activity corresponding to different portions of the brain for a more extensive measure of brain activity and which parts of the brain are active at any given time. For example in some embodiments 4, 6 or more separate electrodes are used providing different electrical signals as input to the processor with each signal corresponding to a different portion of the brain.

In some embodiments the processor 48 is configured to control the head mounted display device 51 to display one or more images to a user of the device and then control the plurality of light emitting elements 40, 42 to emit one or more different predetermined sequences of lights intended to induce a desired neurological response. A user may be instructed to close his or her eyes before the lights are activated, e.g., after being shown a soothing image or video sequence. The system 100 includes separate left 44 and right 46 eye lenses which are placed between the portion of the display corresponding to the users left eye and portion of the display corresponding to the user's right eye. By displaying different images to the left and right eyes, a 3D effect can be, and sometimes is, simulated. Thus, a user can have a 3D virtual reality experience of a scene or environment such as a beach while listening to soothing sounds or inspirations mantras. The memory 46 can be, and sometimes is, used to store videos, audio and/or control routines which when executed by the processor 48 control the system 100 to operate in accordance with the invention.

In some embodiments the electrodes 20, 22 are coupled to the first processor 48 or a second processor, e.g., a processor of a laptop or other device external to the head mount 10. In the case of an external processor being supplied with the detected brain electrical signals detected by the electrodes 20, 22, the signals can be supplied via interface 12 which is coupled to the bus 50 via input/out I/O interface 30. The processor to which the electrodes are coupled and which received the signals indicative of brain activity is configured to monitor electrical signals indicative of brain activity and provide the user of the device one of audio or visual feedback indicative of a detected neurological state. In some embodiments the system 100 is a standalone system which can be worn on the head of a user. The system 100 includes memory 46 for storing videos, light control information and/or detected responses to stimulus that was provided to a user. In some embodiments the memory 46 stores a virtual reality cognitive training program including a 3D video and audio program as well as instructions for controlling the system after the 3D video is presented. In some embodiment when the cognitive training program is executed by the processor 48 of the system 100, the processor 48 causes the display 51 to display 3D video content to a user after which the processor causes the speakers 16, 16′ to output a prompt to the user to close the user's eyes. After a period of time provided to allow the user to close the user's eyes or after receiving user input indicating the user has closed his eyes, e.g., provided by a touch control 70 or another input device, the processor controls the light emitting devices 42, 40 to provide visual stimulation to the user's left and right eyes.

The left and right eye stimulation can be provided simultaneously or independently, e.g., with one eye being stimulated at a time depending on the desired stimulus. By controlling the LED light in a preprogrammed sequence while the user's eyes are closed optical stimulus is provided to the user, e.g., while his eyes are closed. While in some embodiments the stimulus is provided while the eyes are closed, in other embodiments the stimulus is provided while the user's eyes are open. The electrodes 20, 22 are used to measure the user's response to the visual and/or audio program as well as during the subsequent stimulus period. The electrodes 20, 22 detect electrical signals indicating brain activity. These signals are interpreted by the processor 48 and/or an external processor which then provides feedback based on the detected mental state of the user. The feedback is in the form of audio and/or visual feedback to the user indicative of the mental state determined based on the detected electrical signals. Based on the feedback the user can attempt to alter his state of mind by changing his thoughts in an attempt to reach the desired mental state. Alternatively the light pattern emitted by the LEDs 42, 40 may be changed in an attempt to alter the neurological stimulus in an attempt to alter the user's mental state to change it towards a desired mental state. In some embodiments, memory 46 stores a virtual reality cognitive training program which when executed by the processor, e.g., processor 48 of the system causes the processor to display, e.g., on display 51, video content to a user, prompt the user to close the user's eyes, provide visual stimulation by controlling LED light(s), e.g., LEDs 42, 40, in a preprogrammed sequence while the user's eyes are closed, measure the user's response to the provided stimulus by detecting electrical signals using electrodes, e.g., electrodes 22, 20 and provide audio and/or visual feedback to the user indicative of a mental state determined based on the detected electrical signals.

### Numbered List of Exemplary System Embodiments

System Embodiment 1 A system (100) for providing stimulation to a user and detecting user responses to said stimulation, the system (100) comprising: a head mount (10); a plurality of light emitting elements ((42′, 42″, . . . , 42′″), (40′, 40″, . . . 40′″)); a housing (18) secured to said head mount (10); and at least one or more electrodes (22, 20) for making contact with skin of a user.

System Embodiment 2 The system (100) of System Embodiment 1, further comprising: a first processor (48), said first processor (48) being configured to control the plurality of light emitting elements ((42′, 42″, . . . , 42′″), (40′, 40″, . . . 40″′)) to display a sequence of lights during a first period of time.

System Embodiment 3 The system (100) of System Embodiment 3, wherein said lighting elements ((42′, 42″, . . . , 42″′), (40′, 40″, . . . 40′″)) are mounted in said housing (18).

System Embodiment 4 The system (100) of System Embodiment 1, further comprising: an audio output device (16 or 16′) secured to the head mount (10).

System Embodiment 5 The system (100) of System Embodiment 4, further comprising: a head mounted display device (51).

System Embodiment 6 The system (100) of System Embodiment 5 wherein said lighting elements ((42′, 42″, . . . , 42″′), (40′, 40″, . . . 40″′)) include a first set of lighting elements (42) arranged around a left eye lens (44) and a second set of lighting elements (40) arranged around a right eye lens (46).

System Embodiment 7 The system (100) of System Embodiment 6, where the left and right eye lenses (44, 46) are positioned over different portions of said display (51); wherein said first set of lighting elements (42) includes lighting elements (42′, 42″, 42″) of multiple different colors arranged in a circle around said left eye lens (44); wherein said second set of lighting elements (40) includes lighting elements (40′, 40″, 40″) of multiple different colors arranged in a circle around said right eye lens (46).

System Embodiment 8 The system (100) of System Embodiment 7, wherein said lighting elements ((42′, 42″, . . . , 42″′), (40′, 40″, . . . 40″′)) are individual light emitting diodes; and wherein said multiple different colors include two or more of: red, green, or blue.

System Embodiment 9 The system (100) of System Embodiment 5, wherein said processor (48) is further configured to: i) control said head mounted display device (51) to display one or more images to a user of the system (100); and ii) control the plurality of light emitting elements ((42′, 42″, . . . , 42″), (40′, 40″, . . . 40′″)) to emit a sequence of lights intended to induce a desired neurological response.

System Embodiment 10 The system (100) of System Embodiment 9, wherein the electrodes (22, 20) are coupled to the first processor (48) or a second processor (1048), and wherein the processor (48 or 1048) to which the electrodes (22, 20) are coupled is configured to monitor electrical signals indicative of brain activity and provide the user of the system (100) one of audio or visual feedback indicative of a detected neurological state.

System Embodiment 11 The system (100) of System Embodiment 9, wherein the system (100) is a standalone system which can be worn on the head of a user.

System Embodiment 12 The system (100) of System Embodiment 11, wherein said system (100) includes memory (46) for storing videos, light control information and/or detected response to stimulus provided to a user.

System Embodiment 13 The system (100) of System Embodiment 12, wherein said memory (46) stores a virtual reality cognitive training program which when executed by the first processor (48) of the system (100) causes the system (100) to display video content to a user, prompt the user to close the user's eyes, provide visual stimulation by controlling the LED light in a preprogrammed sequence while the user's eyes are closed, measure the user's response to the provided stimulus by detecting electrical signals using the electrodes (22, 20), and provide at least one of audio feedback or visual feedback to the user indicative of a mental state determined based on the detected electrical signals.

System Embodiment 14 The system (100) of System Embodiment 13, wherein said first processor (48) is further configured to determine the mental state of the user based on electrical signals detected by said electrodes (22, 20) which are sensitive to electrical impulses generated by the brain of said user of said system (100).

### Numbered List of Exemplary Method Embodiments

Method Embodiment 1 A method comprising: using a head mounted display to display image content to a user; prompting the user to close the user's eyes; providing visual stimulation by controlling LED light in a preprogrammed sequence while the user's eyes are closed; and measuring the user's response to the provided stimulus by detecting electrical signals using electrodes.

Method Embodiment 2 The method of Method Embodiment 1, wherein said electrodes are part of the wearable system including said headmounted display; and wherein first and second electrodes contact different sides of the user's forehead providing separate electrical channels used to monitor the user's brain activity.

Method Embodiment 3 The method of Method Embodiment 2, wherein measuring the user's response to the provided stimulus includes processing electrical signals detected by said first and second electrodes.

Method Embodiment 4 The method of Method Embodiment 3, further comprising: providing audio or visual feedback to the user indicative of a mental state determined based on the detected electrical signals.

Method Embodiment 5 The method of Method Embodiment 4, wherein providing audio or visual feedback to the user indicative of a mental state determined based on the detected electrical signals includes varying an audio signal output as a function of a first electrical signal detected by said first electrode.

Method Embodiment 6 The method of Method Embodiment 4, wherein providing audio or visual feedback to the user indicative of a mental state determined based on the detected electrical signals includes varying a visual output as a function of a first electrical signal detected by said first electrode, said visual output being an image or color displayed on said head mounted display or one or more lights mounted in said head mounted display, said one or more lights being separate from said visual display.

### Numbered List of Exemplary Computer Readable Medium Embodiments

Computer Readable Medium Embodiment 1 A non-transitory computer readable medium, e.g., memory 46 or memory 1046, including computer executable instructions which when executed by one or more processors, e.g., processor 48 and/or 1048, of a system, e.g., system 100, cause the system to perform the steps of: using a head mounted display, e.g., display 51 or display 1051, to display image content to a user; prompting the user to close the user's eyes; providing visual stimulation by controlling LED light in a preprogrammed sequence while the user's eyes are closed; and measuring the user's response to the provided stimulus by detecting electrical signals using electrodes, e.g., electrodes 20, 22.

Computer Readable Medium Embodiment 2 The non-transitory computer readable medium of Computer Readable Medium Embodiment 1, wherein said electrodes are part of the wearable system including said headmounted display; and wherein first and second electrodes contact different sides of the user's forehead providing separate electrical channels used to monitor the user's brain activity.

Computer Readable Medium Embodiment 3 The non-transitory computer readable medium of Computer Readable Medium Embodiment 2, wherein measuring the user's response to the provided stimulus includes processing electrical signals detected by said first and second electrodes.

Method Embodiment 4 The non-transitory computer readable medium of Computer Readable Medium Embodiment 3, further comprising computer executable instructions which when executed by one or more processors of the system cause the system to perform the steps of: providing audio or visual feedback to the user indicative of a mental state determined based on the detected electrical signals.

Method Embodiment 5 The non-transitory computer readable medium of Computer Readable Medium Embodiment 4, wherein providing audio or visual feedback to the user indicative of a mental state determined based on the detected electrical signals includes varying an audio signal output as a function of a first electrical signal detected by said first electrode.

Method Embodiment 6 The non-transitory computer readable medium of Computer Readable Medium Embodiment 4, wherein providing audio or visual feedback to the user indicative of a mental state determined based on the detected electrical signals includes varying a visual output as a function of a first electrical signal detected by said first electrode, said visual output being an image or color displayed on said head mounted display or one or more lights mounted in said head mounted display, said one or more lights being separate from said visual display.

Computer readable medium embodiment 1—A non-transitory computer readable medium including processor executable instructions for controlling a system including a headmounted display to: operate the head mounted display to display image content to a user; prompt the user to close the user's eyes; provide visual stimulation by controlling LED light in a preprogrammed sequence while the user's eyes are closed; and measure the user's response to the provided stimulus by detecting electrical signals using electrodes.

While steps are shown in an exemplary order it should be appreciated that in many cases the order of the steps may be altered without adversely affecting operation. Accordingly, unless the exemplary order of steps is required for proper operation, the order of steps is to be considered exemplary and not limiting.

Some embodiments are directed a non-transitory computer readable medium embodying a set of software instructions, e.g., computer executable instructions, for controlling a computer or other device, e.g., a headmounted VR device. Other embodiments are directed to a computer readable medium embodying a set of software instructions, e.g., computer executable instructions, for controlling a computer or other device, e.g., a headmounted VR device.

The techniques of various embodiments may be implemented using software, hardware and/or a combination of software and hardware. Various embodiments are directed to apparatus, e.g., a system for providing stimulation to a user and detecting user responses to said stimulation. Various embodiments are also directed to methods, e.g., a method of controlling a head mounted device, providing stimulation to a user, and/or detecting user responses to stimulation. Various embodiments are also directed to a non-transitory machine, e.g., computer, readable medium, e.g., ROM, RAM, CDs, hard discs, etc., which include machine readable instructions for controlling a machine to implement one or more steps of a method.

Various features of the present invention are implemented using components. Such components may, and in some embodiments are, implemented as software components, e.g., software modules. In other embodiments the components are implemented in hardware. In still other embodiments the components are implemented using a combination of software and hardware. In some embodiments the components are implemented as individual circuits with each component being implemented as a circuit for performing the function to which the component corresponds. A wide variety of embodiments are contemplated including some embodiments where different components are implemented differently, e.g., some in hardware, some in software, and some using a combination of hardware and software. It should also be noted that routines and/or subroutines, or some of the steps performed by such routines, may be implemented in dedicated hardware as opposed to software executed on a general purpose processor. Such embodiments remain within the scope of the present invention. Many of the above described methods or method steps can be implemented using machine executable instructions, such as software, included in a machine readable medium such as a memory device, e.g., RAM, floppy disk, etc. to control a machine, e.g., general purpose computer with or without additional hardware, to implement all or portions of the above described methods. Accordingly, among other things, the present invention is directed to a machine-readable medium including machine executable instructions for causing a machine, e.g., processor and associated hardware, to perform one or more of the steps of the above-described method(s).

In some embodiments each of the steps of the described method is performed by a processor or under the control of a processor. Various features address technical problems of how to encode and/or communicate video of a communications network such as the Internet.

Various features address technical problems relating to how to implement a simple to use device which can provide various forms of stimulus, detect neurological responses and provide feedback based on the detected neurological responses and/or detect electrical signals indicative of brain activity.

Numerous additional variations on the methods and apparatus of the various embodiments described above will be apparent to those skilled in the art in view of the above description. Such variations are to be considered within the scope.

What is claimed is:

1. A method for detecting stimulation response from a user, comprising:
displaying, by a head mounted device worn by a user, one or more images, wherein the one or more images are not visible to the user when displayed;
providing visual stimulation by the head mounted device while the user's eyes are closed;
detecting a user response to the visual stimulation based on a measurement by one or more electrodes of the head mounted device;
determining a mental state of the user based on the user response; and
presenting feedback information to the user indicative of the determined mental state.

2. The method of claim 1, wherein displaying the one or more images comprises:
prompting a user to close the user's eyes, and
displaying the one or more images in accordance with determining the user's eyes are closed.

3. The method of claim 1, wherein providing the visual stimulation comprises:
controlling LED light in a preprogrammed sequence while the user's eyes are closed.

4. The method of claim 1, wherein the visual stimulation comprises a left eye visual stimulation and a right eye visual stimulation, and wherein the left eye visual stimulation differs from the right eye stimulation.

5. The method of claim 1, wherein each of the one or more electrodes comprises:
an embedded analog amplifier for amplifying a low level detected brain wave signal to produce an amplified brainwave signal, and
an embedded A/D circuit for converting the amplified received brainwave signal to a digital signal.

6. The method of claim 1, wherein determining the mental state of the user comprises:
comparing the measurements to stored measurement profile information corresponding to a plurality of user mental states.

7. The method of claim 1, further comprising:
determining a stimulation adjustment based on the user response.

8. A non-transitory computer readable medium comprising computer readable code executable by one or more processors to:
display, by a head mounted device worn by a user, one or more images, wherein the one or more images are not visible to the user when displayed;
provide visual stimulation by the head mounted device while the user's eyes are closed;
detect a user response to the visual stimulation based on a measurement by one or more electrodes of the head mounted device;
determine a mental state of the user based on the user response; and
present feedback information to the user indicative of the determined mental state.

9. The non-transitory computer readable medium of claim 8, wherein the computer readable code to display the one or more images comprises computer readable code to:
prompting a user to close the user's eyes, and
displaying the one or more images in accordance with determining the user's eyes are closed.

10. The non-transitory computer readable medium of claim 8, wherein the computer readable code to provide the visual stimulation comprises computer readable code to:
control LED light in a preprogrammed sequence while the user's eyes are closed.

11. The non-transitory computer readable medium of claim 8, wherein the visual stimulation comprises a left eye visual stimulation and a right eye visual stimulation, and wherein the left eye visual stimulation differs from the right eye stimulation.

12. The non-transitory computer readable medium of claim 8, wherein each of the one or more electrodes comprises:
an embedded analog amplifier for amplifying a low level detected brain wave signal to produce an amplified brainwave signal, and
an embedded A/D circuit for converting the amplified received brainwave signal to a digital signal.

13. The non-transitory computer readable medium of claim 8, wherein the computer readable code to determine the mental state of the user comprises computer readable code to:
compare the measurements to stored measurement profile information corresponding to a plurality of user mental states.

14. The non-transitory computer readable medium of claim 8, further comprising computer readable code to:
determine a stimulation adjustment based on the user response.

15. A system comprising:
one or more processors; and
one or more computer readable media comprising computer readable code executable by the one or more processors to:
display, by a head mounted device worn by a user, one or more images, wherein the one or more images are not visible to the user when displayed;
provide visual stimulation by the head mounted device while the user's eyes are closed;
detect a user response to the visual stimulation based on a measurement by one or more electrodes of the head mounted device;
determine a mental state of the user based on the user response; and
present feedback information to the user indicative of the determined mental state.

16. The system of claim 15, wherein the computer readable code to display the one or more images comprises computer readable code to:
prompting a user to close the user's eyes, and
displaying the one or more images in accordance with determining the user's eyes are closed.

17. The system of claim 15, wherein the computer readable code to provide the visual stimulation comprises computer readable code to:
control LED light in a preprogrammed sequence while the user's eyes are closed.

18. The system of claim 15, wherein the visual stimulation comprises a left eye visual stimulation and a right eye visual stimulation, and wherein the left eye visual stimulation differs from the right eye stimulation.

19. The system of claim 15, wherein each of the one or more electrodes comprises:
an embedded analog amplifier for amplifying a low level detected brain wave signal to produce an amplified brainwave signal, and
an embedded A/D circuit for converting the amplified received brainwave signal to a digital signal.

20. The system of claim 15, wherein the computer readable code to determine the mental state of the user comprises computer readable code to:
compare the measurements to stored measurement profile information corresponding to a plurality of user mental states.

21. The system of claim 15, further comprising computer readable code to:
determine a stimulation adjustment based on the user response.
